(19) United States
(12) Patent Application Publication
(10) Pub. No.: US 2015/0119963 A1
(43) Pub. Date: Apr. 30, 2015

(54) MECHANICALLY BISTABLE THERMAL DEVICES

(71) Applicant: Christopher C. Cosse, Shreveport, LA (US)

(72) Inventor: Christopher C. Cosse, Shreveport, LA (US)

(21) Appl. No.: 14/528,010

(22) Filed: Oct. 30, 2014

Related U.S. Application Data
(60) Provisional application No. 61/897,731, filed on Oct. 30, 2013.

(51) Int. Cl. F24J 1/00; F25D 5/02; A47G 19/22; A61F 7/03

(52) U.S. Cl. CPC F24J 1/00; A61F 7/03; F25D 5/02; A47G 19/2288; A61F 2007/0225
USPC 607/112; 126/263.08; 62/4; 222/146.1

(57) ABSTRACT

Mechanically bistable thermal devices and methods of utilizing the same. The mechanically bistable thermal devices include a mechanically bistable element and a thermal element. The mechanically bistable element is configured to be selectively transitioned between a straight conformation, in which the mechanically bistable element defines an elongate shape, and a wrapped conformation, in which the mechanically bistable element defines an arcuate shape and is configured to at least partially encircle an encircled form. The thermal element is configured to exchange thermal energy with the encircled form when in thermal contact with the encircled form and includes a thermal material that includes a fluid and/or a granular solid. The methods include activating the thermal element and transitioning the mechanically bistable element from the straight conformation to the wrapped conformation to at least partially encircle the encircled form with the mechanically bistable device.

### RELATED APPLICATION

This application claims priority to U.S. Provisional Patent Application No. 61/897,731, which was filed on Oct. 30, 2013, and the complete disclosure of which is hereby incorporated by reference.

### FIELD OF THE DISCLOSURE

The present disclosure relates to mechanically bistable thermal devices and to methods that include the mechanically bistable thermal devices.

### BACKGROUND OF THE DISCLOSURE

Thermal devices (e.g., cooling or heating devices) may be placed in thermal contact with a body and may be utilized to heat and/or cool the body. For example, thermal devices may be applied near the skin to relieve pain, swelling, and localized injury. Thermal devices may be used for personal comfort and/or for regulating body temperature, in particular when used next to pulse points (for cooling) or next to extremities (for heating). As another example, thermal devices may be applied to a beverage container and/or may be utilized to heat and/or cool (or maintain a high and/or low temperature of) a beverage that may be contained within the beverage container.

While such thermal devices may be effective at heating and/or cooling a contacted body, it may be difficult and/or time-consuming to retain the thermal devices in contact with the body. As an example, it may be desirable for an athlete to locate and maintain a thermal device in contact with the athlete's skin while the athlete is participating in an athletic event. As another example, it may be desirable to quickly and efficiently retain a thermal device in contact with a beverage container. Thus there exists a need for mechanically bistable thermal devices, and for methods that utilized the mechanically bistable thermal devices.

### SUMMARY OF THE DISCLOSURE

Mechanically bistable thermal devices, and methods for utilizing the same. The mechanically bistable thermal devices include a mechanically bistable element and a thermal element. The mechanically bistable element is configured to be selectively transitioned between a straight conformation, in which the mechanically bistable element defines an elongate shape, and a wrapped conformation, in which the mechanically bistable element defines an arcuate shape and is configured to at least partially encircle an encircled form. The thermal element is configured to exchange thermal energy with the encircled form when in thermal contact with the encircled form, and the thermal element includes a thermal material that includes a fluid and/or granular solid.

The mechanically bistable thermal devices may include and/or be a thermal therapy device that is configured to at least partially encircle and transfer thermal energy with a human body part. The thermal therapy device further may include an actuator that is configured to initiate a chemical interaction within the thermal element. The chemical interaction may include a phase change, a chemical dissolution, a chemical mixing, and/or a chemical reaction. The thermal therapy device may initiate the chemical interaction when, and/or responsive to, the mechanically bistable element transitioning from the straight conformation to the wrapped conformation.

The methods may include methods of operating the mechanically bistable thermal device. The methods include activating the thermal element and transitioning the mechanically bistable element from the straight conformation to the wrapped conformation to at least partially encircle the encircled form with the mechanically bistable device. The activating may be performed before, concurrent with, as a result of, and/or after the transitioning. The activating may initiate thermal energy transfer between the thermal element and the encircled form.

### BRIEF DESCRIPTION OF THE DRAWINGS

FIG. 1 is a schematic representation of examples of a mechanically bistable thermal device, according to the present disclosure, in a straight conformation.

FIG. 2 is a schematic representation of examples of a mechanically bistable thermal device, according to the present disclosure, in a wrapped conformation.

FIG. 3 is a schematic representation of a mechanically bistable element that may be utilized with the devices and methods according to the present disclosure.

FIG. 4 is a fragmentary, partial schematic cross-sectional view of mechanically bistable thermal devices according to the present disclosure.

FIG. 5 is a less schematic side perspective view of a mechanically bistable thermal device according to the present disclosure.

FIG. 6 is a less schematic top plan view of mechanically bistable thermal devices according to the present disclosure.

FIG. 7 is a less schematic top plan view of mechanically bistable thermal devices according to the present disclosure.

FIG. 8 is a less schematic top plan view of mechanically bistable thermal devices according to the present disclosure.

FIG. 9 is a less schematic view of a mechanically bistable thermal device, according to the present disclosure, in the form of a bracelet.

FIG. 10 is a less schematic front view of a mechanically bistable thermal device, according to the present disclosure, in the form of a headband.

FIG. 11 is a less schematic side view of the headband of FIG. 10.

FIG. 12 is a less schematic view of a mechanically bistable thermal device, according to the present disclosure, in the form of a beverage wrap.

FIG. 13 is a less schematic cross-sectional view of a mechanically bistable thermal device, according to the present disclosure, in the form of a beverage wrap.

FIG. 14 is a less schematic cross-sectional view of a mechanically bistable thermal device, according to the present disclosure, in the form of a beverage wrap.

FIG. 15 is a flowchart depicting methods, according to the present disclosure, of managing thermal energy within an encircled form.

### DETAILED DESCRIPTION OF THE DISCLOSURE

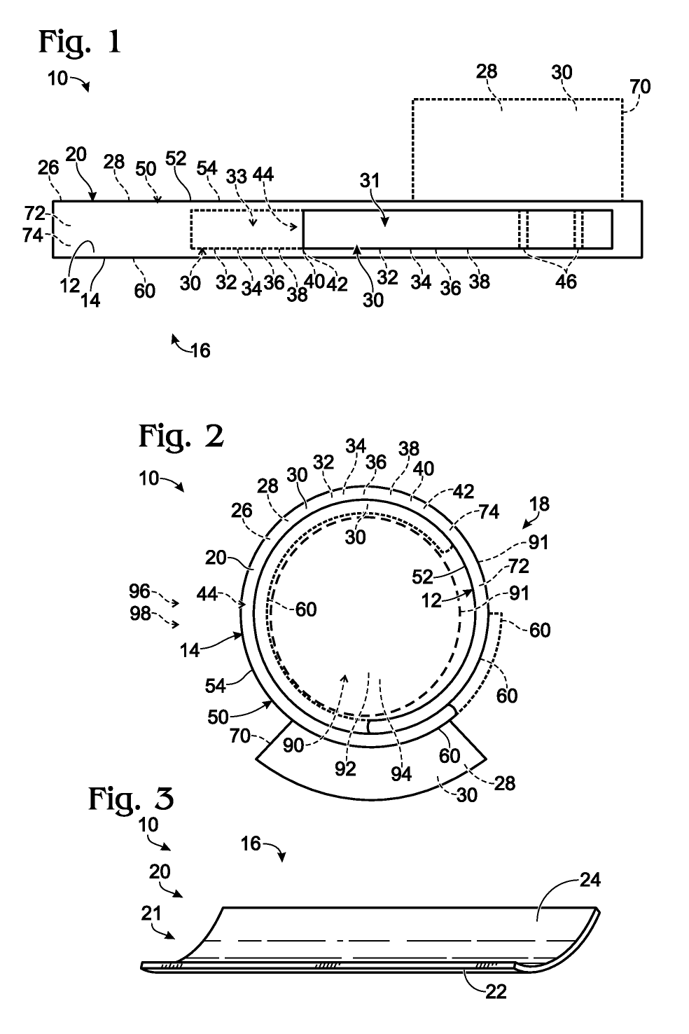

FIGS. 1-15 provide examples of mechanically bistable thermal devices 10 according to the present disclosure, of components of mechanically bistable thermal devices 10, and/or of methods of operating and/or utilizing mechanically bistable thermal devices 10. Elements that serve a similar, or at least substantially similar, purpose are labeled with like numbers in each of FIGS. 1-15, and these elements may not be discussed in detail herein with reference to each of FIGS. 1-15. Similarly, all elements may not be labeled in each of FIGS. 1-15, but reference numerals associated therewith may be utilized herein for consistency. Elements, components, and/or features that are discussed herein with reference to one or more of FIGS. 1-15 may be included in and/or utilized with any of FIGS. 1-15 without departing from the scope of the present disclosure.

In general, elements that are likely to be included in a given (i.e., a particular) embodiment are illustrated in solid lines, while elements that are optional to a given embodiment are illustrated in dashed lines. However, elements that are shown in solid lines are not essential to all embodiments, and an element shown in solid lines may be omitted from a given embodiment without departing from the scope of the present disclosure.

FIG. 1 is a schematic representation of examples of a mechanically bistable thermal device 10, according to the present disclosure, in a straight conformation 16, while FIG. 2 is a schematic representation of examples of mechanically bistable thermal device 10 in a wrapped conformation 18. Mechanically bistable thermal devices 10 also may be referred to herein as thermal devices 10 and/or as devices 10. Devices 10 include a mechanically bistable element 20 and a thermal element 30. Mechanically bistable element 20 also may be referred to herein as bistable element 20 and/or as element 20 and is configured to be selectively transitioned between straight conformation 16 of FIG. 1 and wrapped conformation 18 of FIG. 2. When mechanically bistable element 20 is in straight conformation 16, mechanically bistable thermal device 10 defines and/or has an elongate shape with a longitudinal axis in the elongated (maximum) dimension. When mechanically bistable element 20 is in wrapped conformation 18, mechanically bistable thermal device 10 defines and/or has an arcuate shape and/or is adapted, configured, sized, and/or shaped to at least partially surround, cover, contain, and/or encircle an encircled form 90 (as illustrated in FIG. 2).

Thermal element 30 also may be referred to herein as element 30 and is configured to transfer thermal energy with encircled form 90 when the thermal element is in thermal contact with the encircled form. Devices 10 may be configured such that, when mechanically bistable element 20 is in wrapped conformation 18, thermal element 30 is maintained and/or retained in thermal contact with the encircled form. When in the wrapped conformation, device 10 may extend sufficiently around the encircled form for regions of the device to overlap, thereby at least partially encircling the encircled form with at least two layers of the device.

As illustrated in FIGS. 1-2, device 10 may have and/or define an inner side 12 and an outer side 14. When device 10 is in wrapped conformation 18 of FIG. 2, inner side 12 may be proximal, directed toward, and/or in (direct, physical, and/or thermal) contact with encircled body 90. In contrast, outer side 14 may be distal, directed away from, and/or may not be in (direct, physical, and/or thermal) contact with encircled body 90.

Mechanically bistable element 20 may include any suitable structure that may be adapted, configured, designed, sized, shaped, and/or constructed to have and/or define two stable states, or conformations, including straight conformation 16 and wrapped conformation 18. As an example, and as illustrated in FIG. 3, bistable element 20 may include and/or be an elongate bistable band 21. Elongate bistable band 21 also may be referred to herein as a bistable band 21 and may have and/or define an element inner side 22 and an element outer side 24.

Bistable bands 21 generally are flat, elongated structures that are configured as bistable springs. Bistable bands 21 typically include one or more layered flexible bands composed of a resilient material such as a metal and/or a plastic. In one stable conformation (i.e., straight conformation 16 of FIGS. 1 and 3), the bistable band is generally straight with a slight concavity transverse to the elongated direction. In the other stable conformation (i.e., wrapped conformation 18 of FIG. 2), the bistable band is generally curved, arcuate, circular, and/or spiral-shaped; and the concavity of the straight conformation is largely absent. Bistable bands 21 may be transitioned (or may automatically transition) from the straight conformation to the wrapped conformation via application of an external transition force. For example, this external transition force may be applied by tapping or otherwise striking the inner side of the bistable band when the bistable band is in the straight conformation. This process may be referred to herein as a wrap transition.

A wrap transition may be rapid. For example, bistable element 20 and/or bistable band 21 may rapidly move under its own bias to the wrapped conformation upon receipt of the external transition force that urges the bistable band out of its straight conformation. Once in the wrapped conformation, bistable band 21 is biased to remain in the wrapped conformation until an external straightening force is applied thereto (such as via fully unwrapping and straightening the bistable band into the straight conformation). Bistable band 21 may include and/or may be composed of a thermally conductive component, a thermally insulating component, and/or an electrically conductive component.

Stated another way, bistable element 20 and/or bistable band 21 may have, include, and/or define a first low-energy (or stable) state (i.e., straight conformation 16) and a second low-energy (or stable) state (i.e., wrapped conformation 18). The first low-energy state may be different from the second low-energy state. Additionally or alternatively, the first low-energy state may have a higher potential energy than the second low-energy state.

Bistable element 20 and/or bistable band 21 may be configured to remain in straight conformation 16 of FIGS. 1 and 3 unless urged from the straight conformation via application of the external transition force. Similarly, bistable element 20 and/or bistable band 21 also may be configured to remain in wrapped conformation 18 of FIG. 2 unless urged from the wrapped conformation via application of the external straightening force. A magnitude of the external transition force may be less than a magnitude of the external straightening force and/or an amount of work performed to transition bistable element 20 from straight conformation 16 to wrapped conformation 18 may be less than an amount of work performed to transition bistable element 20 from wrapped conformation 18 to straight conformation 16.

Bistable element 20 may be adapted, configured, sized, constructed, and/or shaped to at least partially conform to an external surface 91 of encircled form 90 when bistable element 20 is in wrapped conformation 18 (as illustrated in FIG. 2). Thus, bistable element 20 may retain thermal element 30 in contact with encircled form 90 when the mechanically bistable element is in the wrapped conformation and may adjust to changes in a shape of external surface 91, as discussed in more detail herein.

It is within the scope of the present disclosure that device 10 may include any suitable number of mechanically bistable elements 20 and that these mechanically bistable elements may be arranged in any suitable manner within device 10. As an example, device 10 may include a plurality of mechanically bistable elements 20 including a primary bistable element 26 and a secondary bistable element 28. Primary bistable element 26 and secondary bistable element 28 may be arranged with any suitable relative orientation within device 10 and are discussed in more detail herein.

Thermal element 30 may include any suitable structure that may be adapted, configured, designed, and/or constructed to transfer thermal energy with the encircled form when the thermal element is in thermal contact with the encircled form. As an example, element 30 may be configured to provide (localized) cooling of the encircled form. As another example, element 30 additionally or alternatively may be configured to provide (localized) heating of the encircled form. Thermal element 30 may be configured to provide the localized heating or cooling at any suitable rate, or degree.

This heating and/or cooling of the encircled form by thermal element 30 may be accomplished in any suitable manner. As an example, thermal element 30 may be configured to undergo an endothermic process to cool (or withdraw thermal energy from) the encircled form. As another example, thermal element 30 also may be configured to undergo an exothermic process to heat (or provide thermal energy to) the encircled form. When thermal element 30 is configured to undergo an endothermic or exothermic process to provide localized cooling or heating, respectively, the thermal element may be configured to undergo this process at a rate and/or at an intensity to provide a suitable degree of cooling or heating. For example, some thermal elements may be configured to undergo, once actuated, a reaction at a relatively constant rate and thereby produce a relatively constant cooling or heating output. As another example, some thermal elements may be configured to undergo, once actuated, a reaction at a rate that slows after an initial period but still continues for at least a second period to provide continued cooling or heating, to maintain a temperature established during the initial period, etc.

Some thermal elements 30 may include a mechanism, or control, that enables a user to adjust (i.e., increase or decrease) the rate of reaction, such as by adjusting the rate at which reactants are mixed and/or otherwise permitted to be in contact with each other. As an example, when the thermal element utilizes oxygen from the ambient air as a reactant, the thermal device may enable a user to adjust the size and/or open/closed state of one or more inlets through which air may contact the reactants within the thermal element.

It is within the scope of the present disclosure that thermal element 30 may be configured (or even required) to be activated and/or actuated prior to transferring (or being capable of transferring) the thermal energy with the encircled form. As an example, thermal element 30 may be activated by being heated and/or cooled. As another example, thermal element 30 may be configured to be selectively actuated, such as via an actuator 44, prior to transferring the thermal energy with the encircled form.

As a more specific example, actuator 44 may be configured to selectively initiate a chemical interaction within thermal element 30. Examples of the chemical interaction include any suitable phase change, chemical dissolution, chemical mixing, and/or chemical reaction. Actuator 44 may be actuated in any suitable manner. As an example, actuator 44 may be manually actuated, such as by a user. As another example, actuator 44 may be automatically actuated, such as via device 10 transitioning from straight conformation 16 of FIGS. 1 and 3 to wrapped conformation 18 of FIG. 2 (i.e., undergoing a wrap transition).

Actuator 44 may include and/or be any suitable structure. As an example, actuator 44 may include and/or be a partition 40 that may separate two different materials, that, when mixed, undergo the endothermic or exothermic interaction. Under these conditions, actuation of actuator 44 may include breaking partition 40 and/or permitting the two different materials to mix.

Device 10 may include both mechanically bistable element 20 and thermal element 30 in any suitable manner. As an example, thermal element 30 may be at least partially defined by, and/or may form a portion of, mechanically bistable element 20. As another example, thermal element 30 may be separate and/or spaced apart from element 20 but operatively attached to element 20 to form device 10.

Mechanically bistable element 20 and thermal element 30 also may have any suitable relative orientation and/or configuration. This is illustrated in FIG. 4, which is a partial schematic fragmentary cross-sectional view of mechanically bistable thermal devices 10 of FIGS. 1-2. As illustrated in FIG. 4 and discussed herein, mechanically bistable element 20 may have and/or define an inner side 22 and an outer side 24, with inner side 22 being directed toward encircled form 90 when device 20 is in wrapped conformation 18 (as illustrated in FIG. 2). As also illustrated in FIG. 4, thermal element 30 may be proximal to, adjacent, and/or operatively attached to inner side 22 and/or outer side 24. Additionally or alternatively, thermal element 30 also may extend between inner side 22 and outer side 24. When thermal element 30 is proximal inner side 22, the thermal element also may be referred to herein as extending between mechanically bistable element 20 and encircled form 90 (when present). Conversely, when thermal element 30 is proximal to outer side 24, the mechanically bistable element also may be referred to herein as extending between thermal element 30 and encircled form 90 (when present).

Returning to FIG. 2, when mechanically bistable element 20 is in wrapped conformation 18, thermal element 30 may be (or be shaped to be) in contact with encircled form 90 (when present). As an example, thermal element 30 may at least partially conform to external surface 91 of encircled form 90. As another example, thermal element 30 may thermally contact encircled form 90 and/or external surface 91 thereof. As yet another example, thermal element 30 may physically contact encircled form 90 and/or external surface 91 thereof.

As illustrated in FIGS. 1-2 and 4, thermal element 30 may include a thermal material 32. Thermal material 32 may include any suitable solid (which may be a granular solid) and/or fluid. The solid and/or fluid may be adapted, configured, synthesized, and/or selected to exchange thermal energy with encircled form 90. The fluid may include any suitable liquid, gas, and/or gel. More specific examples of thermal material 32 include any suitable thermal media 34, phase change material 36, and/or chemical interactant 38.

When thermal material 32 includes thermal media 34, the thermal media may be configured to be thermally treated to a media temperature that is different from a temperature of encircled form 90 prior to contact with encircled form 90. This temperature differential then may provide a motive force to the exchange of thermal energy between the thermal media and the encircled form upon thermal contact therebetween. Examples of thermal media 34 include any suitable high heat capacity material and/or high density material. More specific examples of thermal media 34 include water, a thermal gel, a superabsorbent polymer, and/or glycerin.

When thermal material 32 includes phase change material 36, the phase change material may be configured to undergo a phase change when actuated. This phase change may be endothermic and/or exothermic and may provide a motive force for the exchange of thermal energy between the phase change material and the encircled form subsequent to thermal contact therebetween. Examples of phase change material 36 include a sodium sulfate solution and/or a sodium chloride solution. When thermal material 32 includes phase change material 36, actuator 44 may include and/or be a material that generates nucleation sites upon mechanical deformation thereof. As an example, actuator 44 may be a flexible (or ductile) metallic coupon that may be bent by a user to initiate nucleation of phase change material 36.

When thermal material 32 includes chemical interactant 38, the chemical interactant may be configured to undergo a chemical interaction when activated. This chemical interaction may be endothermic and/or exothermic and may provide a motive force for the exchange of thermal energy between the chemical interactant and the encircled form subsequent to thermal contact therebetween. Examples of the chemical interaction include any suitable chemical reaction, solvation process, and/or chemical mixing process. Chemical interactants 38 generally include systems of two or more materials that may be combined to produce the chemical interaction. Examples of these systems include dry ammonium nitrate and water, dry urea and water, and/or iron oxide and oxygen. It is within the scope of the present disclosure that all components of these systems of chemical interactants may be contained within and/or provided by thermal element 30. Under these conditions, thermal element 30 may include two separate and/or distinct regions, including a first region 31 (as illustrated in solid lines in FIG. 1) and a second region 33 (as illustrated in dashed lines in FIG. 1).

However, it also is within the scope of the present disclosure that one or more components of these systems may be separate from thermal element 30, may be provided from an ambient environment that is proximal to the thermal element, and/or may be provided by a user to initiate the chemical interaction. As an example, the water and/or the oxygen may be separate from thermal element 30, may be provided from the ambient environment, and/or may be provided by the user.

As illustrated in dashed lines in FIGS. 1-2 and 4, device 10 further may include a covering 50. Covering 50 may cover, envelope, surround, and/or enclose mechanically bistable element 20 and/or thermal element 30 and/or may operatively attach mechanically bistable element 20 and thermal element 30. Covering 50 may include and/or be an inner covering 52 and/or an outer covering 54.

When covering 50 includes inner covering 52, thermal element 30 may extend between the inner covering and mechanically bistable element 20 and/or may be associated with (or form) inner side 12 of device 10. Additionally or alternatively, the inner covering may be positioned on device 10 to extend between the encircled form 90 and mechanically bistable element 20 and/or thermal element 30 of the device (as illustrated in FIG. 2). In such a configuration, the inner covering additionally or alternatively may be described as being positioned on device 10 to separate bistable element 20 and/or thermal element 30 from an enclosed form when the device is in its wrapped conformation around the enclosed form. Inner covering 52 may be thermally conductive, such as to facilitate thermal energy transfer between thermal element 30 and encircled form 90. Additionally or alternatively, inner covering 52 also may be adapted, configured, designed, shaped, and/or selected to absorb water, to wick water away from encircled form 90, to promote water evaporation, and/or to repel water.

When covering 50 includes outer covering 54, thermal element 30 may extend between the outer covering and mechanically bistable element 20 and/or may be associated with (or form) outer side 14 of device 10. Additionally or alternatively, mechanically bistable element 20 and/or thermal element 30 may extend between encircled form 90 and the outer coating. Outer covering 54 may be thermally insulating, such as to insulate device 10 from the ambient environment and/or to decrease thermal energy transfer between device 10 and/or encircled form 90 and the ambient environment. Similar to inner covering 52, outer covering 54 may be adapted, configured, designed, shaped, and/or selected to absorb water, to wick water away from encircled form 90, to promote water evaporation, and/or to repel water.

As illustrated in dashed lines in FIGS. 1-2, device 10 also may include a visual indicator 72. Examples of visual indicator 72 include any suitable reflector, printing, luminescent material, fluorescent material, and/or luminous material. As an example, visual indicator 72 may be included and/or selected to increase visibility of device 10 under low-light conditions.

As also illustrated in dashed lines in FIGS. 1-2, device 10 may include an electronic device 74. Examples of electronic device 74 include any suitable RFID tag, electronic monitoring device, electronic therapy device, and/or electronic display device.

As further illustrated in dashed lines in FIGS. 1-2, device 10 may include an extension element 70. Extension element 70 may extend from device 10 (i.e., project away from the elongated length thereof and/or longitudinal axis thereof), may extend from mechanically bistable element 20, may extend past mechanically bistable element 20, and/or may extend in a direction that is perpendicular to a longitudinal axis of mechanically bistable element 20. As discussed in more detail herein, extension element 70 may include and/or contain secondary bistable element 28 and/or thermal element 30. Additionally or alternatively, and as also discussed in more detail herein, extension element 70 may include and/or be a visor that may be configured to restrict and/or block light passage therethrough. As a further example, extension element 70 may be a flange, or other projection that provides a support for the bottom of a beverage container when device 10 is in its wrapped conformation and is used as a beverage wrap.

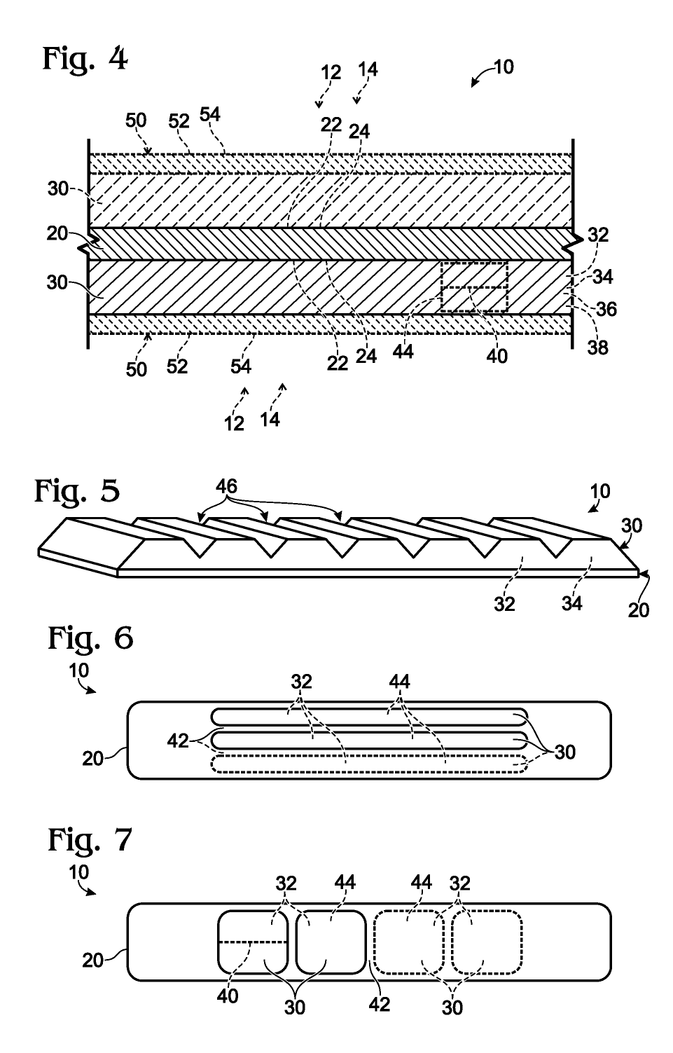

As illustrated schematically in dashed lines in FIG. 1 and less schematically in FIG. 5, thermal element 30 may include one or more relief regions 46. Relief regions 46 may be adapted, configured, sized, and/or constructed to provide space and/or clearance for mechanically bistable element 20 to transition, or more easily transition, between straight conformation 16 and wrapped conformation 18. Additionally or alternatively, relief regions 46 also may be adapted, configured, sized, and/or constructed to increase a flexibility of thermal element 30, thereby facilitating, permitting, and/or allowing mechanically bistable element 20 to transition between the straight conformation and the wrapped conformation. As illustrated in FIG. 5, a device 10 may include a plurality of relief regions 46, such as a plurality of spaced-apart relief regions that extend transverse to the elongated dimension and/or longitudinal axis of the device. The relief region(s) 46, when present, may extend into thermal element 30, may represent thinner regions of thermal element 30, and/or may correspond to regions of less thermal material 32 and/or thermal media 34, as compared to immediately adjacent regions of the device.

Encircled form 90, which is illustrated in FIG. 2, may include any suitable structure that may be at least partially encircled by mechanically bistable element 20 and/or that may exchange thermal energy with thermal element 30. As an example, encircled form 90 may include and/or be a human body part 92. Under these conditions, device 10 also may be referred to herein as a thermal therapy device 96 and may be configured to conform to the human body part when the mechanically bistable element is in the wrapped conformation and/or to align thermal element 30 proximate and/or in contact with the human body part. Examples of the human body part include any suitable wrist, ankle, arm, elbow, leg, knee, palm, sole, head, neck, waist, back, and/or shoulder. Additional examples of thermal therapy devices 96 and/or components thereof are discussed in more detail herein with reference to FIGS. 9-11.

As another example, encircled form 90 may include and/or be a beverage container 94. Under these conditions, device 10 also may be referred to herein as a beverage wrap 98 and may be configured to conform to the beverage container when the mechanically bistable element is in the wrapped conformation. Additional examples of beverage wraps 98 and/or components thereof are discussed in more detail herein with reference to FIGS. 12-14.

Regardless of the exact configuration and/or composition of encircled form 90, device 10 may be configured to adjust to changes in a shape of encircled form 90 and/or may be configured to remain in contact with encircled form 90 despite (or subsequent to) changes in the shape of the encircled form and/or may be configured to contact encircled forms 90 that have a variety of different shapes. As an example, and as illustrated in dashed lines in FIG. 2, device 10 may include an overlap region 60. Under these conditions, a length and/or extent of overlap region 60 may change depending on a shape, size, diameter, effective diameter, and/or cross-sectional area of encircled form 90. As an example, and responsive to an increase in the cross-sectional area of encircled form 90, the extent of overlap region 60 may (automatically) decrease. As another example, and responsive to a decrease in the cross-sectional area of encircled form 90, the extent of overlap region 60 may (automatically) increase.

FIGS. 6-8 are less schematic top view examples of mechanically bistable thermal devices 10 according to the present disclosure. As illustrated in FIG. 6, devices 10 may include a plurality of thermal elements 30 (or elongate thermal elements 30) that may extend along a longitudinal length of device 10 and/or that may be spaced apart from one another in a direction that is perpendicular to the longitudinal length of device 10 (e.g., a transverse direction). As illustrated in FIG. 7, the plurality of thermal elements 30 also may be spaced apart from one another along the longitudinal/elongated length of device 10.

When devices 10 include the plurality of thermal elements 30, individual thermal elements 30 in the plurality of thermal elements 30 may be separated from one another by a seal 42. Seal 42 may be located such that a thermal material 32 that is associated with one thermal element 30 does not (or cannot) contact (or directly contact) a thermal material 32 that is associated with another thermal element 30. As an example, seal 42 of FIG. 6 may extend along the longitudinal length of device 10. As another example, seal 42 of FIG. 7 may extend in the transverse direction.

When devices 10 include the plurality of thermal elements 30, at least a portion of the thermal elements may be configured to be selectively and/or independently actuated. As an example, a first thermal element 30 may be configured to be actuated independent from actuation of a second thermal element 30. As another example, the first thermal element may include a first actuator 44, and the second thermal element may include a second actuator 44 that is separate, distinct, and/or independent from the first actuator.

When devices 10 include the plurality of thermal elements 30, the thermal elements may be configured to heat or cool the encircled form. As an example, each of the plurality of thermal elements may be configured to heat the encircled form. As another example, each of the plurality of thermal elements may be configured to cool the encircled form. As yet another example, the first thermal element may be configured to cool the encircled form, while the second thermal element may be configured to heat the encircled form.

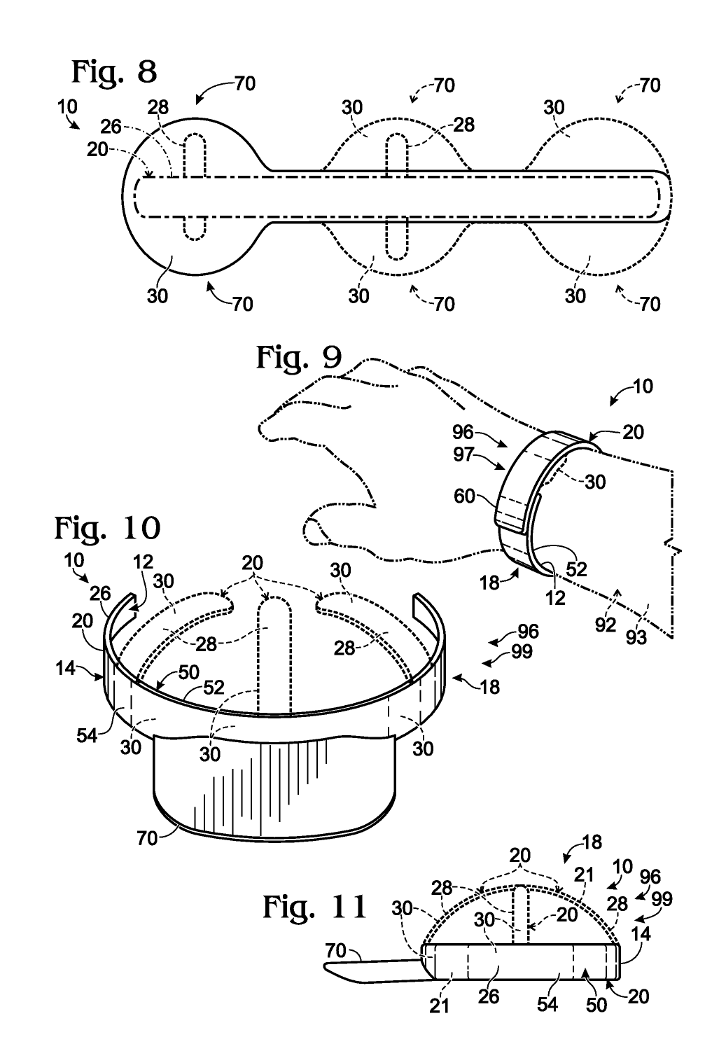

As illustrated in FIG. 8, and discussed herein, devices 10 may include one or more extension elements 70, which may extend in the transverse direction from mechanically bistable element 20 and/or may be wider than the mechanically bistable element. Extension elements 70 may include thermal elements 30, thereby permitting devices 10 that include extension elements 70 to contact, cover, and/or exchange thermal energy with a larger surface area of the encircled form when compared to devices 10 that do not include extension elements 70. Such thermal elements 30, when present, in extension element 70, may be separate from and/or form a portion of a thermal element 30 that extends along and/or in contact with the bistable element. The amount, or concentration, of thermal material 32 and/or thermal media 34 in an extension element 70 may be the same as, less than, or greater than the amount, or concentration, in a corresponding portion of thermal element 30 that extends along and/or in contact with bistable element 20. Additionally or alternatively, extension elements 70 may be formed from an insulating material and may be configured to insulate device 10 and/or the encircled form from the ambient environment.

As also illustrated in FIG. 8, devices 10 that include extension elements 70 may include a primary bistable element 26 and one or more secondary bistable elements 28. Secondary bistable elements 28 may extend within extension elements 70, may be configured to control and/or regulate a shape of extension elements 70, and/or may be configured to retain extension elements 70 in contact with the encircled form.

FIG. 9 is a less schematic view of a mechanically bistable thermal device 10, according to the present disclosure. Device 10 of FIG. 9 is a thermal therapy device 96 in the form of a bracelet 97. In FIG. 9, bracelet 97 is in a wrapped conformation 18 and is shaped to retain a thermal element 30 in contact with a human body part 92, in the form of a wrist 93. Bracelet 97 may be utilized to heat and/or cool wrist 93, which may provide therapeutic treatment of wrist 93. Additionally or alternatively, an athlete may apply bracelet 97 to wrist 93 to assist in regulation of the athlete's body temperature before, during, and/or after physical exertion.

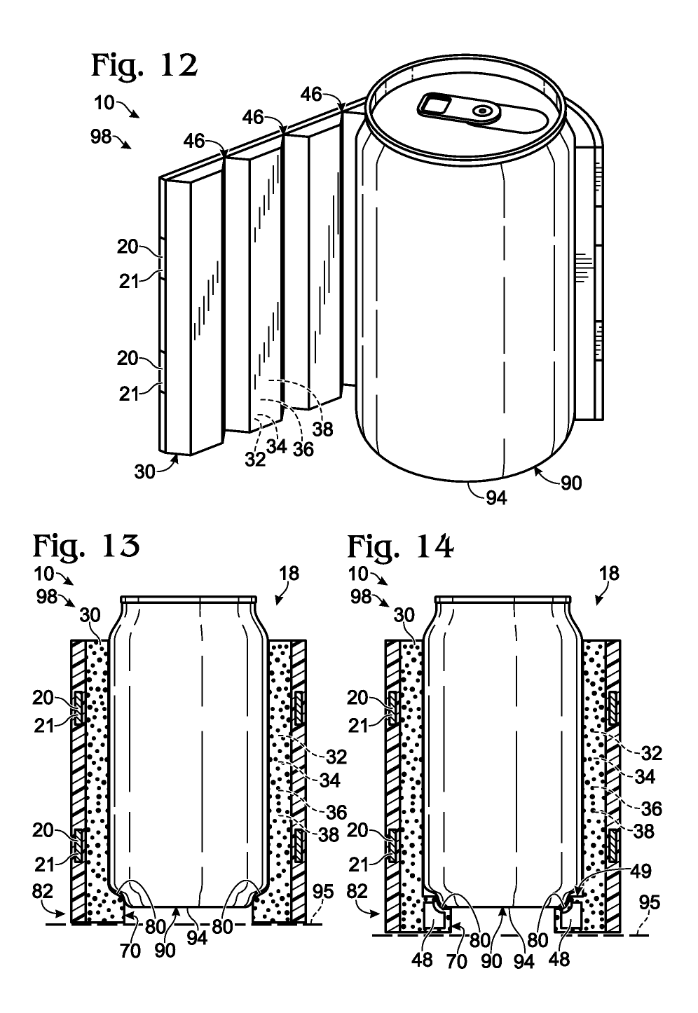

FIGS. 10-11 are less schematic views of a mechanically bistable thermal device 10, according to the present disclosure. Device 10 of FIGS. 10-11 is a thermal therapy device 96 in the form of a headband 99. Headband 99 may be shaped to be placed on and/or to conform to a shape of a user's head and to exchange thermal energy with the user's head. As an example, headband 99 may be utilized to heat the user's head in cold weather. As another example, headband 99 may be utilized to cool the user's head during hot weather and/or during physical exertion.

As illustrated in FIGS. 10-11, headband 99 includes at least one mechanically bistable element 20, and may include a plurality of mechanically bistable elements 20, such as a primary bistable element 26 and one or more secondary bistable elements 28. In the example of FIGS. 10-11, primary bistable element 26 may wrap circumferentially around the user's head, while secondary bistable element(s) 28 may retain headband 99 on the user's head and/or may be utilized to retain headband 99 on top of the user's head. As illustrated, primary bistable element 26 and/or secondary bistable element(s) 28 may be associated with respective thermal elements 30 and may retain the respective thermal elements in contact with the user's head at least when the mechanically bistable elements are in a wrapped conformation 18.

As further illustrated in FIGS. 10-11, headband 99 may include an extension element 70 in the form of a visor. The extension element may be located to protect, shield, and/or shade the user's face and/or eyes. Extension element 70 may project from primary bistable element 26 and/or from an outer side 14 of the primary bistable element.

FIGS. 12-14 are less schematic views of mechanically bistable thermal devices 10, according to the present disclosure, in the form of a beverage wrap 98. Beverage wrap 98 may be adapted, configured, designed, and/or constructed to at least partially encircle and/or conform to an outer surface of an encircled form 90 in the form of a beverage container 94. Beverage wrap 98 may include one or more bistable elements 20, such as one or more mechanically bistable bands 21, and beverage wrap 98 further may include one or more thermal elements 30. The mechanically bistable bands may wrap around and/or encircle the beverage container when the beverage wrap is in a wrapped conformation 18.

As illustrated in FIG. 12, thermal element 30 may include a plurality of relief regions 46, which are discussed in more detail herein. As illustrated in FIGS. 13-14, beverage wrap 98 also may include an extension element 70 in the form of a projection 80 that projects from a side, or edge, region of the device. Projection 80 may extend from beverage wrap 98 and may be configured to retain beverage container 94 within beverage wrap 98, support the bottom of the beverage container, and/or insulate the bottom of the beverage container when the beverage container is at least partially encircled by the beverage wrap.

As also illustrated in FIGS. 13-14, beverage wrap 98 further may include an extension region 82. Extension region 82 may be configured to separate beverage container 94 from a supporting surface 95 when the beverage container is at least partially encircled by the beverage wrap and the beverage container is located on and/or being supported by the supporting surface. As an example, extension region 82 may extend between a bottom surface of the beverage container and the supporting surface.

As illustrated in FIG. 14, beverage wrap 98 also may include a condensate collection region 48 and/or a condensate channel 49. Condensate collection region 48 may be configured to collect a condensate that may form on an outer surface of beverage container 94. Condensate channel 49 may be configured, sized, and/or located to permit the condensate to flow from the outer surface of the beverage container to the condensate collection region.

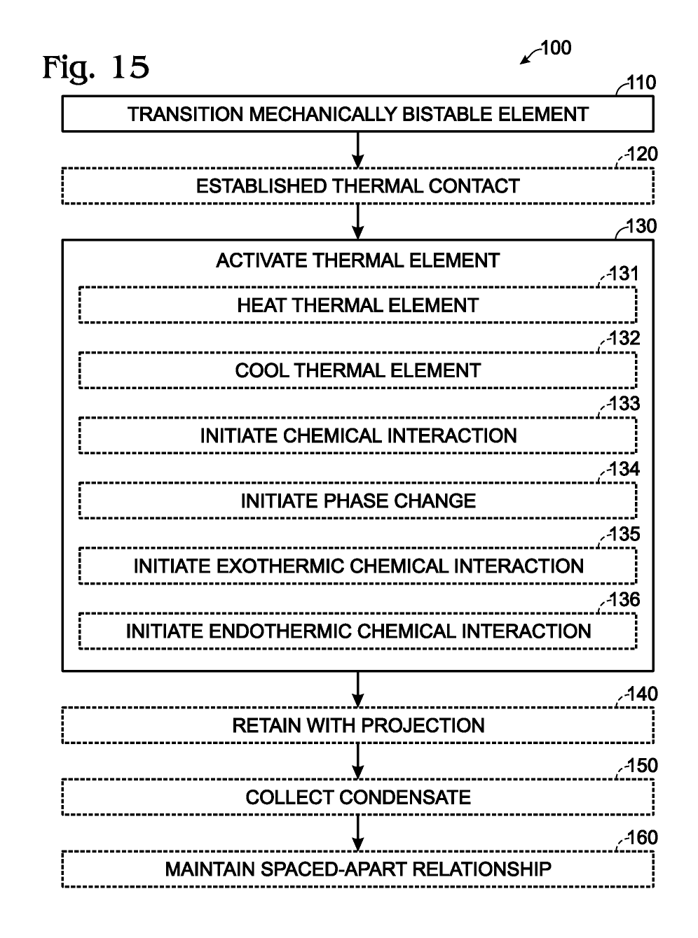

FIG. 15 is a flowchart depicting methods 100, according to the present disclosure, of managing thermal energy within an encircled form. Methods 100 include transitioning a mechanically bistable element at 110 and may include establishing thermal contact between a thermal element and an encircled form at 120. Methods 100 further include activating the thermal element at 130 and may include retaining the thermal element in contact with the encircled form with a projection at 140, collecting condensate at 150, and/or maintaining a spaced-apart relationship between the encircled form and a supporting surface at 160.

Transitioning the mechanically bistable element at 110 may include transitioning any suitable mechanically bistable element, such as mechanically bistable element 20 of any of FIGS. 1-14, from a straight conformation to a wrapped conformation. The transitioning at 110 may include transitioning such that a mechanically bistable thermal device, which includes the mechanically bistable element and the thermal element, at least partially encircles (and optionally fully encircles) the encircled form. As discussed in more detail herein, the transitioning at 110 may include transitioning the mechanically bistable element from a first low-energy state to a second low-energy state and/or performing a wrap transition of the mechanically bistable element.

Establishing thermal contact between the thermal element and the encircled form at 120 may include establishing the thermal contact in any suitable manner. As an example, the establishing at 120 may include establishing direct thermal and/or physical contact between the thermal element and the encircled form. As another example, the establishing at 120 additionally or alternatively may include establishing indirect thermal and/or physical contact between the thermal element and the encircled form. The establishing at 120 may be responsive to, subsequent to, concurrent with, and/or a result of the transitioning at 110.

Activating the thermal element at 130 may include activating the thermal element to permit, facilitate, and/or initiate exchange and/or transfer of thermal energy between the thermal element and the encircled form. The activating at 130 may be an active step that may be performed by a user of the mechanically bistable thermal device and may not be simply based upon, due to, and/or caused by an inherent physical property of the mechanically bistable thermal device.

As an example, the activating at 130 may include heating and/or pre-heating the thermal element, as indicated at 131. This heating and/or pre-heating may be performed prior to the transitioning at 110. As another example, the activating at 130 may include cooling, chilling, and/or pre-chilling the thermal element, as indicated at 132. This cooling, chilling, and/or pre-chilling also may be performed prior to the transitioning at 110.

As yet another example, the activating at 130 may include initiating a chemical interaction within the thermal element, as indicated at 133. Examples of the chemical interaction include any suitable chemical reaction, solvation process, dissolution process, and/or mixing process and are discussed in more detail herein. Generally, the chemical interaction may include combing and/or reacting systems of two or more materials. Examples of such systems also are discussed in more detail herein.

As another example, the activating at 130 may include initiating a phase change within the thermal element and/or within a thermal material that comprises the thermal element, as indicated at 134. As yet another example, the activating at 130 may include initiating an exothermic chemical interaction, or reaction, within the thermal element and/or within the thermal material, as indicated at 135. As another example, the activating at 130 may include initiating an endothermic chemical interaction, or reaction, within the thermal element and/or within the thermal material, as indicated at 136.

It is within the scope of the present disclosure that the activating at 130 may be performed and/or initiated at any suitable time during methods 100 and/or responsive to (or as a result of) any suitable other step and/or action of methods 100. As an example, the activating at 130 may be based upon, concurrent with, responsive to, and/or directly responsive to the transitioning at 110. As another example, the activating at 130 may be independent from, performed prior to, and/or performed subsequent to the transitioning at 110.

It is within the scope of the present disclosure that the mechanically bistable thermal device may include a plurality of thermal elements. Under these conditions, the activating at 130 may include activating at least one (or a first) thermal element of the plurality of thermal elements independently from, separately from, and/or at a different time than at least one other (or a second) thermal element of the plurality of thermal elements. When the mechanically bistable thermal device includes the plurality of thermal elements, each of the plurality of thermal elements may be configured to heat the encircled form or to cool the encircled form. Alternatively, the first thermal element may be configured to heat the encircled form, while the second thermal element may be configured to cool the encircled form.

As discussed herein, the mechanically bistable thermal device may include and/or be a thermal therapy device and the encircled form may include and/or be a human body part. Under these conditions, the transitioning at 110 may include at least partially encircling the human body part with the mechanically bistable thermal device and the activating at 130 may include transferring thermal energy between the thermal element and the human body part. Examples of the human body part are disclosed herein.

As also discussed herein, the mechanically bistable thermal device alternatively may include and/or be a beverage wrap and the encircled form may include and/or be a beverage container. Under these conditions, the transitioning at 110 may include at least partially encircling the beverage container with the mechanically bistable thermal device and the activating at 130 may include transferring thermal energy between the thermal element and the beverage container, such as to regulate a temperature of a beverage that may be contained within the beverage container.

Retaining the thermal element in contact with the encircled form with the projection at 140 may include retaining with any suitable projection that extends from the mechanically bistable thermal device. As an example, and when the mechanically bistable thermal device includes the beverage wrap, the retaining at 140 may include retaining the beverage container within the mechanically bistable thermal device with the projection. As a more specific example, the projection may be sized, shaped, designed, constructed, and/or configured to contact a lower surface of the beverage container and/or to prevent the beverage container from sliding out of the beverage wrap under the influence of gravity.

Collecting condensate at 150 may include collecting condensate that may form on the encircled form. As an example, and when the mechanically bistable thermal device includes the beverage wrap, the beverage may be cooler than an ambient environment that surrounds the beverage and/or the condensate may form on the beverage. Such condensate may be detrimental and/or damaging to a supporting surface upon which the beverage container may be placed. However, the collecting at 150 may be utilized to collect and/or retain the condensate and/or to restrict, or even block, flow of the condensate from the beverage container and onto the supporting surface. This may include collecting with a condensate collection region and/or via a condensate channel, examples of which are disclosed herein.

Maintaining the spaced-apart relationship between the encircled form and the supporting surface at 160 may include maintaining the spaced-apart relationship in any suitable manner. As an example, and when the mechanically bistable thermal device includes the beverage wrap, the maintaining at 160 may include maintaining the beverage container and the supporting surface in a spaced-apart relationship with the mechanically bistable thermal device when the beverage container is at least partially encircled by the beverage wrap and the supporting surface is supporting the beverage wrap and the beverage container. This may include maintaining the spaced-apart relationship with an extension region that extends from the beverage wrap. Examples of the extension region are disclosed herein.

In the present disclosure, several of the examples have been discussed and/or presented in the context of flow diagrams, or flow charts, in which the methods are shown and described as a series of blocks, or steps. Unless specifically set forth in the accompanying description, it is within the scope of the present disclosure that the order of the blocks may vary from the illustrated order in the flow diagram, including with two or more of the blocks (or steps) occurring in a different order and/or concurrently.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities should be understood to mean at least one entity selected from any one or more of the entity in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” may mean A alone, B alone, C alone, A and B together, A and C together, B and C together, A, B and C together, and optionally any of the above in combination with at least one other entity.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

As used herein the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It is also within the scope of the present disclosure that elements, components, and/or other recited subject matter that is recited as being adapted to perform a particular function may additionally or alternatively be described as being configured to perform that function, and vice versa.

As used herein, the phrase, “for example,” the phrase, “as an example,” and/or simply the term “example,” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

Examples of devices and methods according to the present disclosure are presented in the following enumerated paragraphs. It is within the scope of the present disclosure that an individual step of a method recited herein, including in the following enumerated paragraphs, may additionally or alternatively be referred to as a “step for” performing the recited action.

A1. A mechanically bistable thermal device, comprising:

a mechanically bistable element configured to be selectively transitioned between a straight conformation, in which the mechanically bistable element defines an elongate shape, and a wrapped conformation, in which the mechanically bistable element defines an arcuate shape and is configured to at least partially encircle an encircled form; and

a thermal element configured to transfer thermal energy with the encircled form when the thermal element is in thermal contact with the encircled form.

A2. The device of paragraph A1, wherein the mechanically bistable element is an elongate bistable band.

A3. The device of any of paragraphs A1-A2, wherein the straight conformation defines a first low-energy state for the mechanically bistable element, wherein the wrapped conformation defines a second low-energy state for the mechanically bistable element, and further wherein the first low-energy state is different from the second low-energy state, optionally wherein the first low-energy state has a higher potential energy than the second low-energy state.

A4. The device of any of paragraphs A1-A3, wherein, when the mechanically bistable element is in the straight conformation, the mechanically bistable element is configured to remain in the straight conformation unless urged from the straight conformation by an external transition force.

A5. The device of any of paragraphs A1-A4, wherein, when the mechanically bistable element is in the wrapped conformation, the mechanically bistable element is configured to remain in the wrapped conformation unless urged from the wrapped conformation by an external straightening force.

A6. The device of any of paragraphs A1-A5, wherein the mechanically bistable element is biased to automatically transition from the straight conformation to the wrapped conformation responsive to receipt of an/the external transition force.

A7. The device of any of paragraphs A1-A6, wherein the mechanically bistable element is configured to rapidly transition from the straight conformation to the wrapped conformation responsive to receipt of an/the external transition force.

A8. The device of any of paragraphs A1-A7, wherein the mechanically bistable element is configured to retain the thermal element in thermal contact with the encircled form.

A9. The device of any of paragraphs A1-A8, wherein the mechanically bistable element is configured to at least partially conform to an external surface of the encircled form and optionally to adjust to changes in a shape of the external surface of the encircled form.

A10. The device of any of paragraphs A1-A9, wherein the mechanically bistable thermal device includes a plurality of mechanically bistable elements.

A11. The device of any of paragraphs A1-A10, wherein the thermal element is configured to at least one of:

(i) provide localized cooling of the encircled form; and

(ii) provide localized heating of the encircled form.

A12. The device of any of paragraphs A1-A11, wherein the thermal element is configured to at least one of:

(i) undergo an endothermic process to cool the encircled form; and

(ii) undergo an exothermic process to heat the encircled form.

A13. The device of any of paragraphs A1-A12, wherein the thermal element is configured to be activated by cooling and/or heating.

A14. The device of any of paragraphs A1-A13, wherein the thermal element is configured to be selectively actuated.

A15. The device of any of paragraphs A1-A14, wherein the thermal element includes an actuator configured to initiate a chemical interaction, optionally wherein the chemical interaction includes at least one of a phase change, a chemical dissolution, a chemical mixing, and a chemical reaction.

A16. The device of paragraph A15, wherein the mechanically bistable thermal device is configured to initiate the chemical interaction when the mechanically bistable element transitions from the straight conformation to the wrapped conformation.

A17. The device of any of paragraphs A15-A16, wherein the thermal element includes two materials separated by a partition, and wherein the thermal element is configured to initiate the chemical interaction when the partition is broken, optionally wherein the thermal element is configured to break the partition responsive to the mechanically bistable element transitioning from the straight conformation to the wrapped conformation.

A18. The device of any of paragraphs A1-A17, wherein the thermal element forms a portion of the mechanically bistable element.

A19. The device of any of paragraphs A1-A18, wherein the thermal element is separate from and operatively attached to the mechanically bistable element.

A20. The device of any of paragraphs A1-A19, wherein, when the mechanically bistable element is in the wrapped conformation, the mechanically bistable thermal device has an inner side and an outer side, and further wherein the thermal element at least one of:

(i) is adjacent the inner side;

(ii) is adjacent the outer side; and

(iii) extends between the inner side and the outer side.

A21. The device of any of paragraphs A1-A20, wherein, when the mechanically bistable element is in the wrapped conformation, the thermal element is configured to at least one of:

(i) at least partially conform to an/the external surface of the encircled form;

(ii) thermally contact the external surface of the encircled form; and

(iii) physically contact the external surface of the encircled form.

A22. The device of any of paragraphs A1-A21, wherein the thermal element includes a relief region, and optionally a plurality of relief regions, configured to provide clearance for the mechanically bistable element to transition between the straight conformation and the wrapped conformation.

A23. The device of any of paragraphs A1-A22, wherein the thermal element is wider than the mechanically bistable element.

A24. The device of any of paragraphs A1-A23, wherein the thermal element includes a thermal material.

A25. The device of paragraph A24, wherein the thermal material includes at least one of a solid, a granular solid, a fluid, a liquid, and a gel.

A26. The device of any of paragraphs A24-A25, wherein the thermal material includes a thermal media.

A27. The device of paragraph A26, wherein the thermal media is configured to be thermally treated to a media temperature that is different from a temperature of the encircled form and to exchange thermal energy with the encircled form upon thermal contact with the encircled form.

A28. The device of any of paragraphs A26-A27, wherein the thermal media includes a high heat capacity material.

A29. The device of any of paragraphs A26-A28, wherein the thermal media includes a high density material.

A30. The device of any of paragraphs A26-A29, wherein the thermal media includes at least one of water, a thermal gel, a superabsorbent polymer, and glycerin.

A31. The device of any of paragraphs A24-A30, wherein the thermal material includes a phase change material.

A32. The device of paragraph A31, wherein the phase change material is configured to undergo a phase change to exchange thermal energy with the encircled form.

A33. The device of any of paragraphs A31-A32, wherein the phase change material includes at least one of a sodium sulfate solution and a sodium chloride solution.

A34. The device of any of paragraphs A24-A33, wherein the thermal material includes a chemical interactant.

A35. The device of paragraph A34, wherein the chemical interactant is configured to at least one of:

(i) undergo a chemical reaction to exchange thermal energy with the encircled form;

(ii) undergo a solvation process to exchange thermal energy with the encircled form; and

(iii) undergo a chemical mixing process to exchange thermal energy with the encircled form.

A36. The device of any of paragraphs A34-A35, wherein the chemical interactant includes at least one of:

(i) dry ammonium nitrate and water;

(ii) dry urea and water; and

(iii) iron oxide and optionally oxygen.

A37. The device of any of paragraphs A1-A36, wherein the mechanically bistable thermal device comprises a plurality of thermal elements.

A38. The device of paragraph A37, wherein each of the plurality of thermal elements is configured to be selectively and independently actuated.

A39. The device of any of paragraphs A37-A38, wherein each of the plurality of thermal elements is configured to cool the encircled form.

A40. The device of any of paragraphs A37-A38, wherein each of the plurality of thermal elements is configured to heat the encircled form.

A41. The device of any of paragraphs A37-A38, wherein at least one thermal element of the plurality of thermal elements is configured to cool the encircled form, and further wherein at least one thermal element of the plurality of thermal elements is configured to heat the encircled form.

A42. The device of any of paragraphs A1-A41, wherein the mechanically bistable thermal device further includes a covering.

A43. The device of paragraph A42, wherein the covering encloses at least one, and optionally both, of the mechanically bistable element and the thermal element.

A44. The device of any of paragraphs A42-A43, wherein the covering includes an inner covering, and further wherein the thermal element extends between the inner covering and the mechanically bistable element.

A45. The device of paragraph A44, wherein the inner covering is thermally conductive.

A46. The device of any of paragraphs A44-A45, wherein the inner covering is configured to at least one of absorb water, wick water away from the encircled form, promote water evaporation, and repel water.

A47. The device of any of paragraphs A42-A46, wherein the covering includes an outer covering, and further wherein the thermal element extends between the outer covering and the mechanically bistable element.

A48. The device of paragraph A47, wherein the outer covering is thermally insulating.

A49. The device of any of paragraphs A47-A48, wherein the outer covering is configured to at least one of absorb water, wick water away from the encircled form, promote water evaporation, and repel water.

A50. The device of any of paragraphs A1-A49, wherein the mechanically bistable thermal device further includes at least one of a visual indicator and an electronic device.

A51. The device of paragraph A50, wherein the visual indicator includes at least one of a reflector, printing, a luminescent material, a fluorescent material, and a luminous material.

A52. The device of any of paragraphs A50-A51, wherein the electronic device includes at least one of an RFID tag, an electronic monitoring device, an electronic therapy device, and an electronic display device.

A53. The device of any of paragraphs A1-A52, wherein the mechanically bistable thermal device is a thermal therapy device, wherein the encircled form is a human body part, and further wherein the thermal therapy device is configured to conform to the human body part when the mechanically bistable element is in the wrapped conformation.

A54. The device of paragraph A53, wherein the human body part includes at least one of a wrist, an ankle, an arm, an elbow, a leg, a knee, a palm, a sole, a head, a neck, a waist, a back, and a shoulder.

A55. The device of any of paragraphs A53-A54, wherein the thermal therapy device is configured as a wrist band.

A56. The device of any of paragraphs A53-A55, wherein the thermal therapy device is configured as a headband.

A57. The device of paragraph A56, further comprising a visor configured to shade the eyes when the thermal therapy device is worn as the headband, optionally wherein the visor projects from an/the outer side of the mechanically bistable element.

A58. The device of any of paragraphs A53-A57, wherein the thermal therapy device is configured to align the thermal element proximate to one or more of a wrist, an ankle, an arm, an elbow, a leg, a knee, a palm, a sole, a head, a neck, a waist, a back, a shoulder, a carotid artery, a temple, and a kidney.

A59. The device of any of paragraphs A1-A52, wherein the mechanically bistable thermal device is a beverage wrap, wherein the encircled form is a beverage container, and further wherein the beverage wrap is configured to conform to the beverage container when the mechanically bistable element is in the wrapped conformation.

A60. The device of paragraph A59, wherein the beverage wrap further includes a projection configured to retain the beverage container within the beverage wrap when the mechanically bistable element is in the wrapped conformation and the beverage container is at least partially encircled by the beverage wrap.

A61. The device of any of paragraphs A59-A60, wherein the beverage wrap further includes an extension region configured to separate the beverage container from a supporting surface when the mechanically bistable element is in the wrapped conformation, the beverage container is at least partially encircled by the beverage wrap, and the beverage container is being supported by the supporting surface, optionally wherein the extension region extends between a bottom surface of the beverage container and the supporting surface.

A62. The device of any of paragraphs A59-A61, wherein the beverage wrap further includes a condensate collection region configured to collect condensate that forms on the beverage container.

A63. A thermally regulated system, comprising:

the mechanically bistable thermal device of any of paragraphs A1-A62; and

the encircled form.

A64. A method of managing thermal energy within an encircled form, the method comprising:

activating the thermal element of the mechanically bistable thermal device of any of paragraphs A1-A62; and

transitioning the mechanically bistable element from the straight conformation to the wrapped conformation to at least partially encircle the encircled form with the mechanically bistable thermal device.

A65. The method of paragraph A64, wherein the activating includes at least one of preheating and pre-chilling the thermal element.

A66. The method of any of paragraphs A64-A65, wherein the activating includes initiating a chemical interaction within the thermal element.

A67. The method of any of paragraphs A64-A66, wherein the activating is responsive to the transitioning.

B1. A method of managing thermal energy within an encircled form, the method comprising:

transitioning a mechanically bistable element from a straight conformation to a wrapped conformation to at least partially encircle the encircled form with a mechanically bistable thermal device that includes the mechanically bistable element and a thermal element;

responsive to the transitioning, establishing thermal contact between the thermal element and the encircled form; and activating the thermal element to initiate thermal energy transfer between the thermal element and the encircled form.

B2. The method of paragraph B1, wherein the activating is (directly) responsive to the transitioning.

B3. The method of paragraph B 1, wherein the activating is independent from the transitioning.

B4. The method of any of paragraphs B1-B3, wherein the activating includes at least one of:

(i) heating the thermal element prior to the transitioning; and

(ii) cooling the thermal element prior to the transitioning.

B5. The method of any of paragraphs B1-B4, wherein the activating includes initiating a chemical interaction (or reaction) within the thermal element.

B6. The method of any of paragraphs B1-B5, wherein the activating includes initiating a phase change of the thermal element.

B7. The method of any of paragraphs B1-B6, wherein the activating includes initiating an exothermic chemical interaction.

B8. The method of any of paragraphs B1-B6, wherein the activating includes initiating an endothermic chemical interaction.

B9. The method of any of paragraphs B1-B8, wherein the transitioning includes transitioning the mechanically bistable element from a first low-energy state to a second low-energy state.

B10. The method of any of paragraphs B1-B9, wherein the mechanically bistable thermal device includes a plurality of thermal elements, and further wherein the method includes activating at least a first thermal element of the plurality of thermal elements independently from at least a second thermal element of the plurality of thermal elements.

B11. The method of any of paragraphs B1-B10, wherein the first thermal element is configured to heat the encircled form, and further wherein the second thermal element is configured to cool the encircled form.

B12. The method of any of paragraphs B1-B11, wherein the encircled form includes a human body part, wherein the transitioning includes at least partially encircling the human body part with the mechanically bistable thermal device, and further wherein the activating includes transferring thermal energy between the thermal element and the human body part.

B13. The method of paragraph B12, wherein the human body part includes at least one of a wrist, an ankle, an arm, an elbow, a leg, a knee, a palm, a sole, a head, a neck, a waist, a back, and a shoulder.

B14. The method of any of paragraphs B1-B13, wherein the encircled form includes a beverage container, wherein the transitioning includes at least partially encircling the beverage container with the mechanically bistable thermal device, and further wherein the activating includes transferring thermal energy between the thermal element and the beverage container to regulate a temperature of a beverage that is contained within the beverage container.

B15. The method of paragraph B14, wherein the method further includes retaining the beverage container within the mechanically bistable thermal device with a projection that extends from the mechanically bistable thermal device.

B16. The method of any of paragraphs B14-B15, wherein the method further includes collecting condensate that forms on the beverage container within a condensate collection region of the mechanically bistable thermal device.

B17. The method of any of paragraphs B14-B16, wherein the method further includes locating the beverage container and the mechanically bistable thermal device on a supporting surface while the beverage container is at least partially encircled by the mechanically bistable thermal device, and further wherein the method includes maintaining the beverage container and the supporting surface in a spaced-apart relationship with the mechanically bistable thermal device.

### INDUSTRIAL APPLICABILITY

The devices and methods disclosed herein are applicable to the sports industry, the recreation industry, the health care industry, and/or the thermal management industries.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. A thermal therapy device, comprising:
a mechanically bistable element configured to be selectively transitioned between a straight conformation, in which the mechanically bistable element defines an elongate shape, and a wrapped conformation, in which the mechanically bistable element defines an arcuate shape and is configured to at least partially encircle a human body part;
a thermal element configured to transfer thermal energy with the human body part when the thermal element is in thermal contact with the human body part; and
an actuator configured to initiate a chemical interaction within the thermal element, wherein the chemical interaction includes at least one of a phase change, a chemical dissolution, a chemical mixing, and a chemical reaction, and further wherein the thermal therapy device is configured to initiate the chemical interaction when the mechanically bistable element transitions from the straight conformation to the wrapped conformation.

2. A mechanically bistable thermal device, comprising:
a mechanically bistable element configured to be selectively transitioned between a straight conformation, in which the mechanically bistable element defines an elongate shape, and a wrapped conformation, in which the mechanically bistable element defines an arcuate shape and is configured to at least partially encircle an encircled form; and
a thermal element configured to transfer thermal energy with the encircled form when the thermal element is in thermal contact with the encircled form, wherein the thermal element includes a thermal material that includes a fluid or a granular solid.

3. The device of claim 2, wherein the straight conformation defines a first low-energy state for the mechanically bistable element, wherein the wrapped conformation defines a second low-energy state for the mechanically bistable element, and further wherein the first low-energy state is different from the second low-energy state.

4. The device of claim 2, wherein the mechanically bistable element is biased to automatically transition from the straight conformation to the wrapped conformation responsive to receipt of an external transition force.

5. The device of claim 2, wherein the mechanically bistable element is configured to retain the thermal element in thermal contact with the encircled form.

6. The device of claim 2, wherein the thermal element is configured to at least one of:
(i) provide localized cooling of the encircled form; and
(ii) provide localized heating of the encircled form.

7. The device of claim 2, wherein the thermal element includes an actuator configured to initiate a chemical interaction, wherein the chemical interaction includes at least one of a phase change, a chemical dissolution, a chemical mixing, and a chemical reaction.

8. The device of claim 7, wherein the mechanically bistable thermal device is configured to initiate the chemical interaction when the mechanically bistable element transitions from the straight conformation to the wrapped conformation.

9. The device of claim 7, wherein the thermal element includes two materials separated by a partition, wherein at least one of the two materials includes the fluid, wherein the thermal element is configured to initiate the chemical interaction when the partition is broken, and further wherein the thermal element is configured to break the partition responsive to the mechanically bistable element transitioning from the straight conformation to the wrapped conformation.

10. The device of claim 2, wherein the thermal element includes at least one relief region configured to provide clearance for the mechanically bistable element to transition between the straight conformation and the wrapped conformation.

11. The device of claim 2, wherein the thermal element includes a thermal material.

12. The device of claim 11, wherein the thermal material includes a thermal media, wherein the thermal media is configured to be thermally treated to a media temperature that is different from a temperature of the encircled form and to exchange thermal energy with the encircled form upon thermal contact with the encircled form.

13. The device of claim 11, wherein the thermal material includes a phase change material configured to undergo a phase change to exchange thermal energy with the encircled form.

14. The device of claim 11, wherein the thermal material includes a chemical interactant configured to at least one of:
(i) undergo a chemical reaction to exchange thermal energy with the encircled form;
(ii) undergo a solvation process to exchange thermal energy with the encircled form; and
(iii) undergo a chemical mixing process to exchange thermal energy with the encircled form.

15. The device of claim 2, wherein the mechanically bistable thermal device comprises a plurality of thermal elements.

16. The device of claim 15, wherein each of the plurality of thermal elements is configured to be selectively and independently actuated.

17. The device of claim 15, wherein at least one thermal element of the plurality of thermal elements is configured to cool the encircled form, and further wherein at least one thermal element of the plurality of thermal elements is configured to heat the encircled form.

18. The device of claim 2, wherein the mechanically bistable thermal device is a thermal therapy device, wherein the encircled form is a human body part, and further wherein the thermal therapy device is configured to conform to the human body part when the mechanically bistable element is in the wrapped conformation.

19. The device of claim 2, wherein the mechanically bistable thermal device is a beverage wrap, wherein the encircled form is a beverage container, and further wherein the beverage wrap is configured to conform to the beverage container when the mechanically bistable element is in the wrapped conformation.

20. The device of claim 19, wherein the beverage wrap further includes a projection configured to retain the beverage container within the beverage wrap when the mechanically bistable element is in the wrapped conformation and the beverage container is at least partially encircled by the beverage wrap.

21. The device of claim 19, wherein the beverage wrap further includes an extension region configured to separate the beverage container from a supporting surface when the mechanically bistable element is in the wrapped conformation, the beverage container is at least partially encircled by the beverage wrap, and the beverage container is being supported by the supporting surface.

22. A method of managing thermal energy within an encircled form, the method comprising:
activating the thermal element of the mechanically bistable thermal device of claim 2; and
transitioning the mechanically bistable element from the straight conformation to the wrapped conformation to at least partially encircle the encircled form with the mechanically bistable thermal device.

23. A method of managing thermal energy within an encircled form, the method comprising:
transitioning a mechanically bistable element from a straight conformation to a wrapped conformation to at least partially encircle the encircled form with a mechanically bistable thermal device that includes the mechanically bistable element and a thermal element;
responsive to the transitioning, establishing thermal contact between the thermal element and the encircled form; and
activating the thermal element to initiate thermal energy transfer between the thermal element and the encircled form.

24. The method of claim 23, wherein the activating is directly responsive to the transitioning.

25. The method of claim 23, wherein the activating is independent from the transitioning.

26. The method of claim 23, wherein the activating includes at least one of:
(i) heating the thermal element prior to the transitioning;
(ii) cooling the thermal element prior to the transitioning;
(iii) initiating a chemical interaction within the thermal element;
(iv) initiating a phase change of the thermal element;
(v) initiating an exothermic chemical interaction; and
(vi) initiating an endothermic chemical interaction.

27. The method of claim 23, wherein the mechanically bistable thermal device includes a plurality of thermal elements, and further wherein the method includes activating at least a first thermal element of the plurality of thermal elements independently from at least a second thermal element of the plurality of thermal elements.

28. The method of claim 23, wherein the encircled form includes a human body part, wherein the transitioning includes at least partially encircling the human body part with the mechanically bistable thermal device, and further wherein the activating includes transferring thermal energy between the thermal element and the human body part.

29. The method of claim 23, wherein the encircled form includes a beverage container, wherein the transitioning includes at least partially encircling the beverage container with the mechanically bistable thermal device, and further wherein the activating includes transferring thermal energy between the thermal element and the beverage container to regulate a temperature of a beverage that is contained within the beverage container.
